(19) United States
(12) Patent Application Publication
(10) Pub. No.: US 2015/0073445 A1
(43) Pub. Date: Mar. 12, 2015

(54) Implantable Porous Device Including a Film

(71) Applicant: Covidien LP, Mansfield, MA (US)

(72) Inventors: Jeremy Griffin, Hamden, CT (US); Amin Elachchabi, Hamden, CT (US); Daniel Broom, Branford, CT (US); Ryan Witherell, Glastonbury, CT (US); Garrett Ebersole, Hamden, CT (US); Joshua Stopek, Minneapolis, MN (US)

(21) Appl. No.: 14/389,829

(22) PCT Filed: Aug. 12, 2013
(86) PCT No.: PCT/US2013/054460

§ 371 Date: Oct. 1, 2014

Related U.S. Application Data
(60) Provisional application No. 61/682,455, filed on Aug. 13, 2012.

(51) Int. Cl. A61F 2/00

(52) U.S. Cl. CPC A61F 2/0063; A61F 2250/0067
USPC 606/151

(57) ABSTRACT

A surgical implant includes a porous substrate defining a length along a longitudinal axis and a width along a transverse axis, and a discontinuous film disposed over a surface of the porous substrate. The discontinuous film defines a plurality of coated segments on the porous substrate that are spaced by uncoated regions of the porous substrate.

### CROSS-REFERENCE TO RELATED APPLICATIONS

This application claims the benefit of and priority to U.S. Provisional Patent Application No. 61/682,455, filed Aug. 13, 2012, the entire disclosure of which is incorporated by reference herein.

### TECHNICAL FIELD

The present disclosure relates generally to medical devices. More particularly, the present disclosure relates to multi-laminar surgical implants.

### BACKGROUND

Techniques for repairing damaged or diseased tissue are widespread in medicine. Wound closure devices, such as sutures and staples, as well as other repair devices, such as mesh or patch reinforcements, may be used to repair tissue defects or injuries, e.g., herniated tissue, prolapses, fistulas, stomas, and other damaged and/or diseased tissue. For example, in the case of hernias, a mesh or patch may be used to reinforce the abdominal wall. The mesh or patch may be generally sized to extend across the defect and adapted to flex or bend to conform to movement of the abdominal wall. The mesh or patch may be held in place by adhering, suturing, or stapling the mesh to the surrounding tissue.

Difficulties, however, may arise during, or after, a hernia repair procedure, such as tearing or breaking of the mesh or patch at the repaired hernia opening. Tearing and breakage may compromise the surgical repair of the hernia defect, or lead to mesh failure.

It would be advantageous to provide a surgical implant including a multi-layered configuration that is strong and resists tearing, yet is supple for maneuverability, folding, and flexing of the surgical implant.

### SUMMARY

A surgical implant of the present disclosure includes a porous substrate defining a length along a longitudinal axis and a width along a transverse axis, and a discontinuous film disposed over a surface of the porous substrate. The discontinuous film defines a plurality of coated segments on the porous substrate that are spaced by uncoated regions of the porous substrate. The coated segments may be arranged in coated regions that form a patterned grouping of coated segments on the porous substrate.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a top view of a surgical implant in accordance with an embodiment of the present disclosure;

FIG. 2A is a perspective view of a surgical implant in accordance with another embodiment of the present disclosure;

FIG. 2B is a close-up top view of a region of the surgical implant of FIG. 2A;

FIG. 3 is a schematic illustration of a targeting feature of a surgical implant in accordance with an embodiment of the present disclosure; and

FIG. 4 is a top view of a surgical implant in accordance with an embodiment of the present disclosure.

### DETAILED DESCRIPTION

Surgical implants in accordance with the present disclosure include a multi-layered structure having a porous substrate and a discontinuous non-porous layer. The porous substrate provides a flexible primary structure to the implant, while the discontinuous non-porous layer reinforces the primary structure and maintains flexibility of the porous layer. More specifically, the discontinuous non-porous layer allows the porous substrate to flex, bend, and/or fold along axes devoid of the non-porous layer to provide multiple planes of maneuverability and folding while limiting the rigidity of the reinforced surgical implant.

While the present discussion and figures below depict a surgical implant in the form of a surgical mesh for hernia repair, the presently disclosed surgical implants can be used in connection with other surgical procedures requiring repair of soft tissue defects such as muscle or wall tissue defects, pelvic organ prolapse, and urinary incontinence, for example, and may be any surgical implant, such as a scaffold, graft, patch, sling, pledget, growth matrix, drug delivery device, wound plug, and, in general, any soft tissue repair device or surgical prosthesis that can be used in medical/surgical procedures. The surgical implant may also be utilized as an externally applied medical product, such as a wound dressing, covering, and gauze, for example.

Porous substrates in accordance with the present disclosure may be a mesh, fibrous sheet, patch, foam, film, or composite thereof. The term “porous” as used herein may define openings and spacings which are present as a surface characteristic or a bulk material property, partially or completely penetrating the substrate. Suitable materials for forming the porous substrate include, but are not limited to fibrous structures (e.g., knitted structures, woven structures, non-woven structures, etc.), foams (e.g., open or closed cell foams), and perforated films. Use of a porous substrate may allow for quicker healing through the openings formed therein.

The porous substrate should have the following characteristics: sufficient tensile strength to support a fascial wall during repair of a defect in the fascial wall causing a hernia; sufficiently inert to avoid foreign body reactions when retained in the body for long periods of time; easily sterilized to prevent the introduction of infection when the substrate is implanted in the body; sufficient pore density, size and distribution to allow for optimal healing and tissue ingrowth; and suitably easy handling characteristics for placement in the desired location in the body. The porous substrate should be sufficiently pliable to conform to a fascial wall and flex with movement of the wall, while being sufficiently rigid to retain its shape. The porous substrate should also be sufficiently strong (e.g., tensile strength) to avoid tearing of portions thereof.

In embodiments, the porous substrate is fabricated from a textile including yarns. Yarns forming the porous substrate may be monofilament or multifilament yarns which may be made of any suitable biocompatible material. In some embodiments, the yarns include at least two filaments which may be arranged to create openings therebetween, the yarns also being arranged relative to each other to form openings in the porous substrate. Alternatively, the porous substrate may be formed from a continuous yarn that is arranged in loops that give rise to the openings in the porous substrate. The use of a porous substrate having yarns spaced apart in accordance with the present disclosure has the advantage of reducing the foreign body mass that is implanted in the body, while maintaining sufficient tensile strength to securely support the defect and tissue being repaired by the porous substrate. Moreover, the openings of the porous substrate of the present disclosure may be sized to permit fibroblast through-growth and ordered collagen laydown, resulting in integration of the porous substrate into the body. Thus, the spacing between the yarns may vary depending on the surgical application and desired implant characteristics as envisioned by those skilled in the art. Moreover, due to the variety of sizes of defects, and of the various fascia that may need repair, the porous substrate may be of any suitable size.

The yarns may be braided, twisted, aligned, fused, or otherwise joined to form a variety of different porous substrate shapes. In embodiments in which at least two filaments form a yarn, the filaments may be drawn, oriented, crinkled, twisted, braided, commingled or air entangled to form the yarn. The yarns may be woven, knitted, interlaced, braided, or formed into a porous substrate by non-woven techniques. The structure of the porous substrate will vary depending upon the assembling technique utilized to form the porous substrate, as well as other factors such as the type of fibers used, the tension at which the yarns are held, and the mechanical properties required of the porous substrate.

In embodiments, knitting may be utilized to form a porous substrate of the present disclosure. Knitting involves, in embodiments, the intermeshing of yarns to form loops or inter-looping of the yarns. In some embodiments, yarns may be warp-knitted thereby creating vertical interlocking loop chains and/or may be weft-knitted thereby creating rows of interlocking loop stitches across the porous substrate. In other embodiments, weaving may be utilized to form a porous substrate of the present disclosure. Weaving may include, in embodiments, the intersection of two sets of straight yarns, warp and weft, which cross and interweave at right angles to each other, or the interlacing of two yarns at right angles to each other. In some embodiments, the yarns may be arranged to form a porous substrate which has isotropic or near isotropic tensile strength and elasticity.

In embodiments, the yarns may be nonwoven and formed by mechanical, chemical, or thermal bonding of the yarns into a sheet or web in a random or systematic arrangement. For example, yarns may be mechanically bound by entangling the yarns to form the porous substrate by means other than knitting or weaving, such as matting, pressing, stitch-bonding, needle-punching, or otherwise interlocking the yarns to form a binderless network. In other embodiments, the yarns of the porous substrate may be chemically bound by use of an adhesive, such as a hot melt adhesive, or thermally bound by applying a binder, such as a powder, paste, or melt, and melting the binder on the sheet or web of yarns.

In embodiments, the porous substrate may be a self-fixating substrate formed from a knit having grip members extending from at least one surface of the porous substrate. In embodiments, the grip members may protrude perpendicularly with respect to the surface of the porous substrate. Examples of suitable grip members include hooks, loops, spiked naps, darts, barbs and combinations thereof. One example of a self-fixation mesh which may be utilized as the porous substrate of the surgical implant of the present disclosure is Parietex Progrip™ Self-fixating Mesh, commercially available from Tyco Heatlthcare Group LP, d/b/a Covidien.

The porous substrate may be fabricated from any biodegradable and/or non-biodegradable polymer that can be used in surgical procedures. The term “biodegradable” as used herein is defined to include both bioabsorbable and bioresorbable materials. By biodegradable, it is meant that the material decomposes, or loses structural integrity under body conditions (e.g., enzymatic degradation or hydrolysis) or is broken down (physically or chemically) under physiologic conditions in the body such that the degradation products are excretable or absorbable by the body. Absorbable materials are absorbed by biological tissues and disappear in vivo at the end of a given period, which can vary for example from hours to several months, depending on the chemical nature of the material. It should be understood that such materials include natural, synthetic, bioabsorbable, and/or certain non-absorbable materials, as well as combinations thereof.

Representative natural biodegradable polymers which may be used to form implants of the present disclosure include: polysaccharides such as alginate, dextran, chitin, chitosan, hyaluronic acid, cellulose, collagen, gelatin, fucans, glycosaminoglycans, and chemical derivatives thereof (substitutions and/or additions of chemical groups include, for example, alkyl, alkylene, amine, sulfate, hydroxylations, carboxylations, oxidations, and other modifications routinely made by those skilled in the art); catgut; silk; linen; cotton; and proteins such as albumin, casein, zein, silk, soybean protein, and copolymers and blends thereof; alone or in combination with synthetic polymers.

Synthetically modified natural polymers which may be used to form implants, and in certain embodiments, yarns include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, and cellulose sulfate sodium salt.

Representative synthetic biodegradable polymers which may be utilized to form implants described herein include polyhydroxy acids prepared from lactone monomers such as glycolide, lactide, caprolactone, ε-caprolactone, valerolactone, and δ-valerolactone, carbonates (e.g., trimethylene carbonate, tetramethylene carbonate, and the like), dioxanones (e.g., 1,4-dioxanone and p-dioxanone), 1,dioxepanones (e.g., 1,4-dioxepan-2-one and 1,5-dioxepan-2-one), and combinations thereof. Polymers formed therefrom include: polylactides; poly(lactic acid); polyglycolides; poly(glycolic acid); poly(trimethylene carbonate); poly(dioxanone); poly(hydroxybutyric acid); poly(hydroxyvaleric acid); poly(lactide-co-(ε-caprolactone-)); poly(glycolide-co-(ε-caprolactone)); polycarbonates; poly(pseudo amino acids); poly(amino acids); poly(hydroxyalkanoate)s such as polyhydroxybutyrate, polyhydroxyvalerate, poly(3-hydroxybutyrate-co-3-hydroxyvalerate), polyhydroxyoctanoate, and polyhydroxyhexanoate; polyalkylene oxalates; polyoxaesters; polyanhydrides; polyester anyhydrides; polyortho esters; and copolymers, block copolymers, homopolymers, blends, and combinations thereof.

Some non-limiting examples of suitable non-degradable materials from which the surgical implants may be made include polyolefins such as polyethylene (including ultra high molecular weight polyethylene) and polypropylene including atactic, isotactic, syndiotactic, and blends thereof; polyethylene glycols; polyethylene oxides; polyisobutylene and ethylene-alpha olefin copolymers; fluorinated polyolefins such as fluoroethylenes, fluoropropylenes, fluoroPEGSs, and polytetrafluoroethylene; polyamides such as nylon, Nylon 6, Nylon 6,6, Nylon 6,10, Nylon 11, Nylon 12, and polycaprolactam; polyamines; polyimines; polyesters such as polyethylene terephthalate, polyethylene naphthalate, polytrimethylene terephthalate, and polybutylene terephthalate; polyethers; polybutester; polytetramethylene ether glycol; 1,4-butanediol; polyurethanes; acrylic polymers; methacrylics; vinyl halide polymers such as polyvinyl chloride; polyvinyl alcohols; polyvinyl ethers such as polyvinyl methyl ether; polyvinylidene halides such as polyvinylidene fluoride and polyvinylidene chloride; polychlorofluoroethylene; polyacrylonitrile; polyaryletherketones; polyvinyl ketones; polyvinyl aromatics such as polystyrene; polyvinyl esters such as polyvinyl acetate; etheylene-methyl methacrylate copolymers; acrylonitrile-styrene copolymers; ABS resins; ethylene-vinyl acetate copolymers; alkyd resins; polycarbonates; polyoxymethylenes; polyphosphazine; polyimides; epoxy resins; aramids; rayon; rayon-triacetate; spandex; silicones; and copolymers and combinations thereof.

The discontinuous non-porous layer may be a discontinuous film disposed over a surface of the porous substrate. The discontinuous film is disposed on the porous substrate, and defines a plurality of coated segments thereon. The discontinuous film may be formed from any of the biodegradable and/or non-biodegradable polymers described above, and may be the same or different from the polymer forming the porous substrate. The discontinuous film may be applied to the porous substrate in a variety of ways, creating a coated segment on the porous substrate. Some examples of methods to create the porous substrate include, but are not limited to, spraying, dipping, layering, casting, calendering, etc. The coated segments may be of the same shape and/or size and disposed at evenly spaced intervals along the porous substrate, or may be provided in a variety of shape, size, and spacing configurations depending upon the required performance characteristics for the envisaged application of use.

The porous substrate and/or discontinuous film may be used to deliver therapeutic agents. In general, therapeutic agents may be incorporated into the porous substrate and/or discontinuous film during manufacture or formation of the porous substrate and/or film, using methods including, but not limited to, free solution, suspension, liposomal delivery, microspheres, etc., coating a surface of the porous substrate and/or discontinuous filmt, or selective regions thereof, such as by polymer coating, dry coating, freeze drying, or applying the coating directly to a surface of the porous substrate and/or discontinuous film. In embodiments, at least one therapeutic agent may be combined with the absorbable porous substrate and/or discontinuous film to provide release of the therapeutic agent via degradation of the surgical implant. The therapeutic agent may be freely admixed with the polymeric material forming the porous substrate and/or discontinuous film, or may be tethered to the polymer through suitable chemical bonds.

Therapeutic agents include any substance or mixture of substances that have clinical use. Consequently, therapeutic agents may or may not have pharmacological activity per se, e.g., a dye. Alternatively, a therapeutic agent could be any agent which provides a therapeutic or prophylactic effect; a compound that affects or participates in tissue growth, cell growth and/or cell differentiation; a compound that may be able to invoke or prevent a biological action such as an immune response; or a compound that could play any other role in one or more biological processes. A variety of therapeutic agents may be incorporated into the surgical implant of the present disclosure. Moreover, any agent which may enhance tissue repair, limit the risk of sepsis, and modulate the mechanical properties of the surgical implant (e.g., the swelling rate in water, tensile strength, etc.) may be added during the preparation of the surgical implant or may be coated thereon.

Examples of classes of therapeutic agents which may be utilized in accordance with the present disclosure include antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, and enzymes. It is also intended that combinations of therapeutic agents may be used.

Other therapeutic agents which may be in the present disclosure include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g., oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; estrogens; antibacterials; antibiotics; anti-fungals; anti-virals; anticoagulants; anticonvulsants; antidepressants; antihistamines; and immunological agents.

Other examples of suitable therapeutic agents which may be included in the present disclosure include: viruses and cells; peptides, polypeptides and proteins, as well as analogs, muteins, and active fragments thereof; immunoglobulins; antibodies; cytokines (e.g., lymphokines, monokines, chemokines); blood clotting factors; hemopoietic factors; interleukins (IL-2, IL-3, IL-4, IL-6); interferons (β-IFN, (α-IFN and γ-IFN)); erythropoietin; nucleases; tumor necrosis factor; colony stimulating factors (e.g., GCSF, GM-CSF, MCSF); insulin; anti-tumor agents and tumor suppressors; blood proteins; gonadotropins (e.g., FSH, LH, CG, etc.); hormones and hormone analogs (e.g., growth hormone); vaccines (e.g., tumoral, bacterial and viral antigens); somatostatin; antigens; blood coagulation factors; growth factors (e.g., nerve growth factor, insulin-like growth factor); protein inhibitors; protein antagonists; protein agonists; nucleic acids such as antisense molecules, DNA, and RNA; oligonucleotides; and ribozymes.

Embodiments of the present disclosure will now be described below while referencing the accompanying figures. The accompanying figures are merely examples and are not intended to limit the scope of the present disclosure.

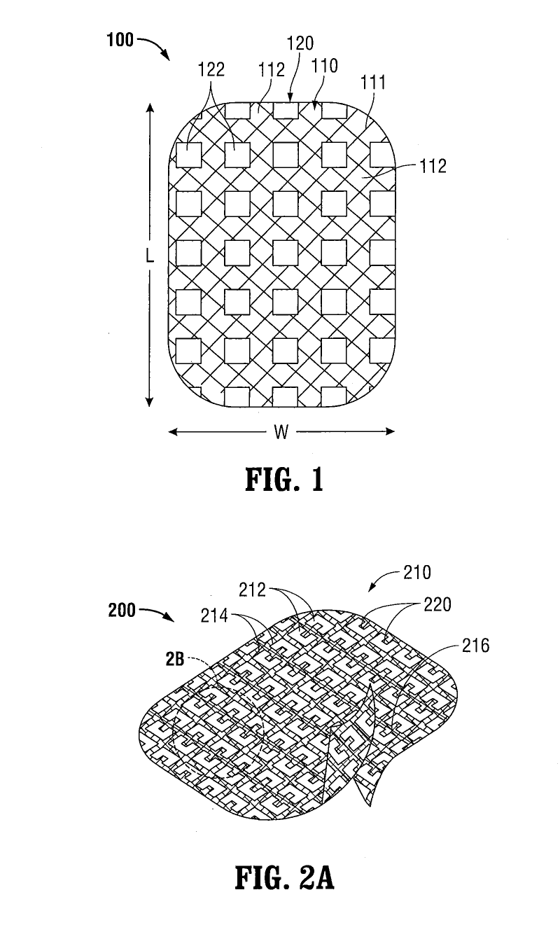

Referring now to the drawings wherein like components are designated by like reference numerals throughout the several views, FIG. 1 illustrates a surgical implant in the form of a surgical mesh 100, according to an embodiment of the present disclosure. Surgical mesh 100 includes a porous substrate 110 formed of a plurality of fibers 111 defining a length along a longitudinal axis “L” and a width along a transverse axis “W”. A discontinuous film 120 is coated thereon a surface of the porous substrate 110. It should be noted that the discontinuous film 120 may be provided only on a portion of a surface of a porous substrate, an entire surface of a porous substrate, or a plurality of surfaces of a porous substrate.

Discontinuous film 120 includes a plurality of coated segments 122 disposed on the porous substrate 110 that are broken up, or spaced, by uncoated regions 112 of the porous substrate 110. As illustrated, the coated segments 122 are similarly sized and shaped, and evenly distributed on the porous substrate 110. While the coated segments 122 are illustrated as squares, it should be understood, that the coated segments may be any shape, for example, other geometric shapes, such as circles, triangles, diamonds, etc.

The discontinuous film 120 improves the tear resistance of the porous substrate 110, while leaving the porous substrate 110 supple and responsive to applied forces. The uncoated regions 112 extend linearly along both the length and width of the porous substrate 110 and define a plurality of axes that are substantially parallel to the longitudinal and transverse axes, L and W, respectively, allowing the surgical implant 100 to bend, fold and/or flex therealong. The coated segments 122 reinforce the porous substrate 110 and minimize or prevent tear propagation in the uncoated regions 112. As illustrated in FIG. 1, at least some of the coated segments 122 are disposed across the pores and/or interstitial spaces positioned between the plurality of fibers 111.

While the uncoated regions 112 are illustrated as extending linearly along an entire length or width of the porous substrate 110, it should be understood that the uncoated regions may extend only partially, non-linearly, and/or in other angular relationships with respect to the longitudinal and transverse axes of the porous substrate, depending upon the pattern of the discontinuous film.

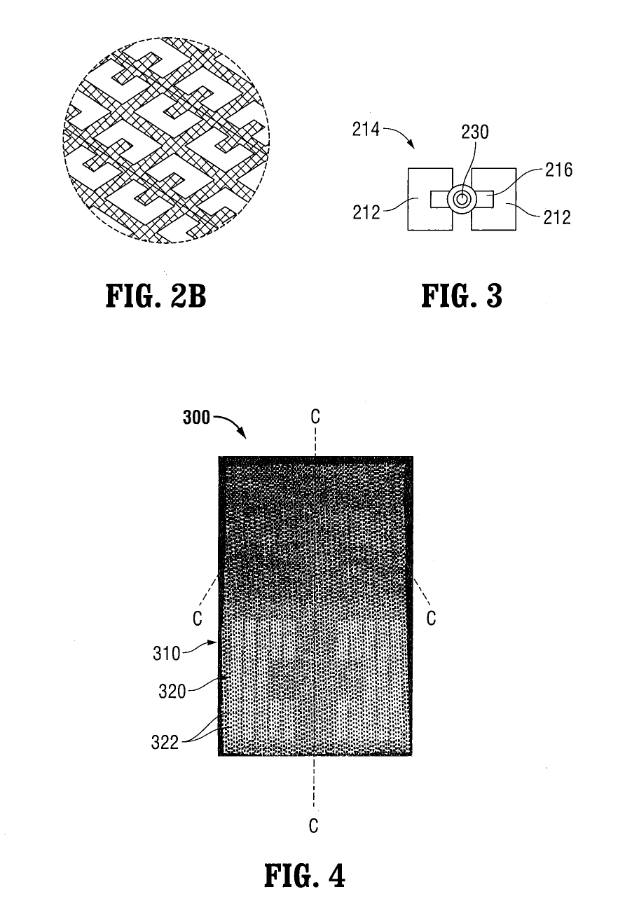

A discontinuous film may include a plurality of coated regions made up of a plurality of coated segments to form a patterned grouping of coated segments on a porous substrate. As illustrated in FIGS. 2A and 2B, surgical mesh 200 includes a porous substrate 210 and a discontinuous film 220 including a plurality of coated regions 214 composed of a pair of coated segments 212. The coated segments 212 are illustrated as complementary, mirror-imaged “C”-shaped brackets that are symmetrically spaced about the porous substrate 210.

In embodiments, a center portion 216 of the coated regions 214 may define a visual target, or pre-determined location, for mesh anchoring of the surgical mesh 200 with a tissue fixation device (e.g., sutures, tacks, staples, etc.). In other embodiments, as illustrated in FIG. 3, for example, a target 230 may be provided in the center portion 216 of the coated regions 214 as markings applied with ink to the porous substrate 210 that may be visualized under visible, infrared, ultraviolet, and/or by other wavelengths of light. In some embodiments, the target 230 may include a therapeutic agent, such as an analgesic, which releases upon penetration of the surgical mesh with a tissue fixation device, thereby providing a local benefit to tissue. Thus, a visual target will allow a clinician to more easily orientate a surgical mesh within a surgical site, properly fasten the surgical mesh against tissue, and/or decrease the pain or discomfort associated with fastening a surgical mesh by providing immediate therapeutic relief to the surgical site.

The orientation of a surgical mesh may also be indicated by the placement of a discontinuous film on a porous substrate. For example, as illustrated in FIG. 4, a surgical mesh 300 may include a discontinuous film 320 coating only a portion of a porous substrate 310. The discontinuous film 320 is provided on about half of the porous substrate 310, providing a clinician with a visual indication of, for example, the medial and outer edges of the surgical mesh 300 to aid in proper positioning of the surgical mesh 300 during implantation. Moreover, placement of the discontinuous film 320 about select portion of the porous substrate 310 may also allows a clinician to perform trimming practices on the surgical mesh 300 without compromising the multi-layered construction of the implant, as illustrated, for example, by cut lines “C”, shown in phantom.

The coated segments 322 of the discontinuous film 320 may form an overall pattern on the porous substrate 310. Thus, in embodiments, the coated segments 322 of the discontinuous film 320 may be utilized for product identification, branding, or for conveying other information about the surgical mesh to a clinician.

As further illustrated in FIG. 4, coated segments 322 are disposed to the individual fibers 311 and are not disposed across the pores and/or interstitial spaces positioned between the plurality of fibers 311. Such a configuration provides the implant with a predetermined folding characteristic without reducing the implants porosity.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments of the present disclosure. Various modifications and variations of the surgical implant will be apparent to those skilled in the art from the foregoing detailed description. Such modifications and variations are intended to come within the scope and spirit of the claims appended hereto.

What is claimed is:

1. A surgical implant comprising:
a porous substrate defining a length along a longitudinal axis and a width along a transverse axis; and
a discontinuous film disposed over a surface of the porous substrate, the discontinuous film defining a plurality of coated segments on the porous substrate, the coated regions being spaced by uncoated regions of the porous substrate.

2. The surgical implant according to claim 1, wherein the coated segments are of substantially similar shape and size.

3. The surgical implant according to claim 1, wherein the coated segments are evenly distributed across the surface of the porous substrate.

4. The surgical implant according to claim 1, wherein the uncoated regions extend linearly along the porous substrate and define a plurality of axes about which the porous substrate may be folded.

5. The surgical implant according to claim 5, wherein the plurality of axes are substantially parallel with the longitudinal axis of the porous substrate.

6. The surgical implant according to claim 4, wherein the plurality of axes are substantially parallel with the transverse axis of the porous substrate.

7. The surgical implant according to claim 1, wherein the coated segments are arranged in a plurality of coated regions to form a patterned grouping of coated segments on the porous substrate.

8. The surgical implant according to claim 7, wherein each coated region comprises a pair of coated segments.

9. The surgical implant of claim 8, wherein the pair of coated segments are complementary, mirror-imaged shapes.

10. The surgical implant of claim 7, wherein the coated regions are evenly distributed across the surface of the porous substrate.

11. The surgical implant of claim 1, further comprising a therapeutic agent.

12. The surgical implant of claim 1, further comprising a target disposed on the surface of the porous layer.

13. The surgical implant of claim 12, wherein the target is defined by two or more coated segments of the discontinuous film.

14. The surgical implant of claim 12, wherein the target are markings on the porous substrate.

15. The surgical implant of claim 12, wherein the target includes a therapeutic agent.
